(19) United States
(12) Patent Application Publication
(10) Pub. No.: US 2012/0109677 A1
(43) Pub. Date: May 3, 2012

(54) BLOOD BANK MANAGEMENT AND RAPID BLOOD TYPE DETERMINATION SYSTEM

(75) Inventor: Christoper D. Hillyer, New York, NY (US)

(73) Assignee: New York Blood Center, Inc., New York, NY (US)

(21) Appl. No.: 13/285,988

(22) Filed: Oct. 31, 2011

Related U.S. Application Data
(60) Provisional application No. 61/408,981, filed on Nov. 1, 2010.

(51) Int. Cl. G06Q 50/22; G01N 33/53

(52) U.S. Cl. 705/2; 435/7.25

(57) ABSTRACT

Disclosed herein is a method for increasing the efficiency and reducing cost of the blood donation, processing and storage by pre-screening candidate donors for blood type and only collecting blood from donors having a needed blood type. Also disclosed are methods and systems for rapid determination of blood type of a blood donor prior to the initiation of the blood donation process.

### CROSS-REFERENCE TO RELATED APPLICATIONS

The present application claims the benefit under 35 U.S.C. §119(e) of U.S. Provisional Application 61/408,981 filed Nov. 1, 2010, the entire contents of which is incorporated by reference herein.

### FIELD

The present disclosure is generally related to the field of blood typing, and particularly to rapid pre-donation blood typing for use in the blood collection and banking industry and methods for screening candidate blood donors.

### BACKGROUND

Red blood cell antigens, called blood groups, are differentially expressed in individuals requiring testing of blood before transfusion to determine compatibility between the donor blood and the recipient. The blood group systems expressed on the surface of red blood cells include, but are not limited to the ABO, Rhesus (Rh), Kell, Duffy, MNS and Lewis systems.

The testing procedures for determining the ABO blood group and Rh type of an individual's blood are well known to those skilled in the art. The major blood group is ABO, which includes group A, group B, group AB and group O. These blood groups are determined by the antigens present on an individual's red blood cells. An individual with group A blood carries red blood cells with type A antigens. In addition, persons of a particular ABO type have antibodies in their blood plasma which react with the antigens that they lack. For example, individuals who are group A have antibodies to the B antigen. Group O individuals have both anti-A and anti-B antibodies. An additional important antigen is the D antigen in the Rh system. Blood typing tests are performed on the person's red blood cells to determine which antigens are present.

In the currently used blood donation process, blood group typing is only performed on donated blood, not on pre-donation samples. Therefore, blood is collected without regard to the donor's blood type. However, at any given time, not all types of blood are needed for a particular blood product. Therefore, a rapid blood typing system is needed to type donors prior to donation, during the screening process, to avoid obtaining undesired types of a particular blood product. This would reduce the collection costs of “less valuable” blood types as well as the additional cost of discarding unused units.

### SUMMARY

Disclosed herein are methods and systems for increasing the efficiency and reducing the cost of blood donation, processing and storage by rapidly screening candidate blood donors for blood type and determining whether the blood type is in demand or is over-stocked. Candidate blood donors with less- or un-desirable blood types can be excused from the blood donation process or offered to donate a non-red cell product (i.e. platelets or plasma by apheresis) to reduce costs and waste.

In one embodiment, a method is provided for increasing the efficiency and reducing cost of blood donation, processing and storage comprising: a) pre-determining at least one blood type needed by a blood collection center; b) determining a blood type of a blood sample from at least one candidate blood donor; and c) acting upon the results of the determining step by an action selected from the group consisting of (i) accepting the candidate blood donor for blood donation if the blood type of the candidate donor corresponds to the blood type needed in step (a), (ii) rejecting the candidate blood donor is the candidate donor's blood type is not needed in step (a), or (iii) referring the candidate donor to donation of an alternate blood product independent of the blood type of the candidate donor.

Also disclosed herein is a method for increasing the efficiency and reducing cost of blood donation, processing and storage comprising: a) pre-determining blood types needed by a blood collection center; b) obtaining a blood sample from at least one candidate blood donor; c) mixing at least a portion of the blood sample with reagents for determining blood types to form a reaction mixture; d) processing the reaction mixture for a time and at a temperature sufficient for a reaction to occur; e) detecting the reaction or absence thereof; f) correlating the reaction or absence thereof with a blood type; and (e) acting upon the results of the correlating step by an action selected from the group consisting of (i) accepting the candidate blood donor for blood donation if the blood type of the candidate donor corresponds to the blood type needed in step (a), (ii) rejecting the candidate blood donor is the candidate donor's blood type is not needed in step (a), or (iii) referring the candidate donor to donation of an alternate blood product independent of the blood type of the candidate donor, such that the efficiency of the blood donation process is increased and the costs of blood donation, processing and storage is reduced.

In another embodiment, the method further comprises the step of pre-determining a blood product needed by a blood collection center.

In another embodiment, the blood product is whole blood or red blood cells. In yet another embodiment, the alternate blood product is platelets or plasma. In another embodiment, the blood type is an ABO blood group type. In yet another embodiment, the blood type is an Rh(D) blood group type.

In another embodiment, the determining step comprises an antibody-based assay. In yet another embodiment, the antibody is an immunoglobulin M (IgM) antibody.

In one embodiment disclosed herein, a method is provided for determining blood type prior to blood donation using a blood type determination system, the method comprising the steps of: (a) obtaining a sample of blood from a candidate blood donor; (b) mixing at least a portion of the blood sample with an antibody specific for a blood group antigen; (c) determining the blood type of the blood based on the agglutination of the red blood cells by the antibody; (d) repeating steps (b) and (c) for each blood group antigen to be tested; and (e) developing a blood type phenotype for the candidate donor based upon the blood type determined in step (d).

In another embodiment disclosed herein, a method is provided for determining blood type prior to blood donation using a blood type determination system, the method comprising the steps of: (a) obtaining a sample of blood from a candidate blood donor; (b) mixing at least a portion of the blood sample with an antibody specific for a blood group antigen; (c) applying the mixture to a reaction vessel, wherein the reaction vessel comprises a filter matrix; (d) centrifuging the reaction vessel to facilitate the passage of non-agglutinated red blood cells through the filter matrix to the bottom of the reaction vessel; and (e) determining the blood type of the blood based on the agglutination of the red blood cells by the antibody; (f) repeating steps (b)-(e) for each blood group antigen to be tested; and (g) developing a blood type phenotype for the candidate donor based upon the blood type determined in step (e).

In another embodiment, the blood type is an ABO blood group type. In yet another embodiment, the blood type is an Rh(D) blood group type.

In another embodiment, the determining step comprises an antibody-based assay. In yet another embodiment, the antibody is an immunoglobulin M (IgM) antibody. In another embodiment, the antibody is a control antibody.

In another embodiment, the blood determination system comprises a plurality of reaction vessels, wherein the reaction vessels further comprise a filter matrix; at least antibody specific for a blood group antigen; and optionally, a centrifuge.

In another embodiment, the plurality of reaction vessels are grouped together on a card or plate. In another embodiment, the filter matrix is an inert material with pores or a size exclusion material. In yet another embodiment, the blood type determination system further comprises negative and positive control reaction vessels.

Also disclosed herein is a system for increasing the efficiency and reducing cost of blood donation, processing and storage in a blood bank or blood collection center comprising an inventory of blood and/or blood component units available in the blood bank or blood collection center; a kit comprising a blood type determination system for determining the blood type of a candidate donor prior to donation; and means for determining if the blood type of the candidate donor is desirable in the inventory.

### BRIEF DESCRIPTION OF THE DRAWINGS

FIG. 1 depicts one embodiment of the blood type determination system for determination of A (FIG. 1A), B (FIG. 1B) and D (FIG. 1C) blood group antigens.

### DETAILED DESCRIPTION

Disclosed herein are methods and systems for increasing the efficiency and reducing the cost of blood donation, processing and storage by rapidly screening candidate blood donors for blood type and determining whether the blood type is in demand or is over-stocked. Candidate blood donors with less- or un-desirable blood types can be excused from the blood donation process to reduce costs and waste or alternately asked to donate plasma or platelets by apheresis.

For the purpose of this disclosure, the term “blood” refers to whole blood, red blood cells and red blood cell products, including automated red blood cell (2RBC) collection. Automated red blood cell collection allows the donor to give two units of red blood cells, instead of just one. The process separates blood into its components while it is being drawn. The remaining components are returned to the donor.

Furthermore, there are various blood products needed by patients including red blood cells, plasma products (including fresh frozen within 24 hours [PF24], fresh frozen plasma [FFP], and cryoprecipitate-reduced plasma), platelets, and cryoprecipitate. Platelets are either isolated from collected units of whole blood and pooled to make a therapeutic dose or collected by apheresis, sometimes concurrently with plasma or red blood cells. Apheresis is a process in which the blood of a donor or patient is passed through an apparatus that separates out one particular constituent and returns the remainder to the circulation, in contrast to whole blood collection, where a pint of blood is collected from a donor and no material is returned to the donor's bloodstream.

The blood type of a recipient governs the blood or blood products suitable for donation to that recipient. Blood group AB individuals have both A and B antigens on the surface of their RBCs and their blood plasma does not contain any antibodies against either A or B antigen. Therefore, an individual with type AB blood can receive blood from any group, but their red cells can only be transfused to another group AB individual and their plasma can be transfused to group O, A, B, or AB individuals (universal donor plasma).

Blood group A individuals have the A antigen on the surface of their RBCs, and their blood plasma contains IgM antibodies against the B antigen. Therefore, a group A individual can receive blood only from individuals of groups A or O (with A being preferable), but their red cells can be transfused to group A or AB individuals and their plasma can be transfused to group A or group O individuals.

Blood group B individuals have the B antigen on the surface of their RBCs, and their blood plasma contains IgM antibodies against the A antigen. Therefore, a group B individual can receive blood only from individuals of groups B or O (with B being preferable), but their red cells can be transfused to group B or AB individuals and their plasma can be transfused to group B or group O individuals.

Blood group O individuals do not have either A or B antigens on the surface of their RBCs, but their blood plasma contains IgG and IgM anti-A, anti-B and anti-A,B antibodies. Therefore, a group O individual can receive blood only from a group O individual, but their red cells can be transfused to group O, A, B, or AB individuals (universal donor red cells) and their plasma can be transfused only to group O individuals.

An Rh(D)-negative patient who does not have any anti-D antibodies (never being previously exposed to (Rh)D-positive RBCs) can receive a transfusion of (Rh)D-positive blood once, but this would cause a sensitization to the D antigen. If a D-negative patient has developed anti-D antibodies, a subsequent exposure to (Rh)D-positive blood would lead to a potentially dangerous transfusion reaction. Of particular risk, in women of child-bearing age who are Rh(D)-negative and has antibodies against D antigen there is significant risk of hemolytic disease of the newborn if the fetus is Rh(D)-positive. Because of this risk, Rh(D)-positive blood is usually not given to (Rh)D-negative women of child bearing potential or to patients with D antibodies, blood banks must conserve Rh(D)-negative blood for these patients. In some circumstances, such as for a major bleed when stocks of Rh(D)-negative blood units are low at the blood bank, Rh(D)-positive blood might be given to Rh(D)-negative females above child-bearing age or to Rh(D)-negative males, providing that they did not have anti-D antibodies, to conserve Rh(D)-negative blood stock in the blood bank. The converse is not true; Rh(D)-positive patients do not react to Rh(D)-negative blood.

This same blood type matching is done for other antigens of the Rh system, such as C, c, E and e, and for other blood group systems with a known risk for immunization, such as the Kell system, in particular for females of child-bearing potential or patients with known need for many transfusions.

Blood group O individuals are also known as universal donors and their blood (containing RBCs) can be administered to anyone in an emergency. Blood group AB individuals are universal donors for plasma and Rh(D) negative individuals are universal donors for whole blood or apheresis (not plasma).

A large number of variables must be managed in order to meet blood collection and inventory goals. Blood collection centers have experienced an increase in the demand for type-specific blood and blood components. Moreover, the supply of donors is inadequate to meet the demand of some blood type, most notably group O, Rh(D)-negative. Therefore systems and methods of aligning blood collection and thus blood supply with blood type demand are needed.

Blood banks and blood collection centers maintain records of blood and blood product inventory and demand and seek to meet demand for blood products of a particular type by preferentially attracting donors of a desired blood type. Typically, any and all candidate blood donors that qualify, based on a questionnaire and minimum health requirements, are processed through the blood donation system. After donation, blood typing and suitability assays (for risk of infectious agents that may be harmful for potential recipients) are performed and blood units that are not suitable are destroyed. Some of these units are destroyed due to the presence of (or evidence of infection with) certain pathogenic agents.

If donor blood is collected, determined to be suitable, and not used by the expiration date (approximately 42 days after collection for red cell products), it is destroyed. An additional 2.3 million units of red cell units were available nationwide in excess of demand in 2009. The majority of these units were destroyed. Many units of blood are destroyed because the donor blood type is not needed and the units of this blood type expire before they can be used. Before these unneeded units are destroyed, they have consumed valuable resources in supplies, storage space and manpower to recruit donors, collect, inventory and maintain the units. Under current practices, no pre-donation blood type screening of candidate donors is conducted which would avoid collecting blood of undesirable or unneeded types. Additionally, there is not a system for directing a donor of a certain blood type to donate the most valuable product which is needed of their blood type.

There is no existing system and method for rapidly blood typing candidate donors and deciding, based on the candidate donor blood type, whether to collect blood from the candidate, to release the candidate without collecting blood, or to collect an alternate product. By more accurately aligning blood donations with blood type, those candidate donors whose blood type is not desirable, or currently needed, can be released and asked to return at a future time, or asked to donate an alternate product by apheresis. Those candidate donors whose blood type is in demand can be more expeditiously passed through the blood donation process, making the process less time consuming and more respectful of the donors' time.

The disclosed methods and systems will more closely align the blood donation process with blood type demands, thereby making the blood donation process more efficient, less costly and less wasteful.

The methods for increasing the efficiency and reducing the cost of blood donation involve an inventory of blood products available at the blood collection center that are aligned with the hospital blood bank, and thus the patient, needs. Efficiency in blood collection will also result in more efficient and less costly blood processing and storage as only needed units will be processed and stored For the purposes of this disclosure, a blood collection center refers to any facility or organization that collects blood from donors such as a blood center, blood bank, hospital, clinic, or other facility or organization that collects blood.

Methods for determining if a candidate blood donor type is desirable or in demand, or to the contrary not desirable for a particular product, in the inventory of a blood collection center include but is not limited to review or analysis of any record or data tracking system such as ledgers, log books, computer databases, etc. In certain embodiments, a blood collection center provides to personnel a daily analysis of available blood types and recommendations of in-demand blood types or those most desirable of increasing inventory. In other embodiments, the blood collection center provides the analysis on an hourly, daily, weekly or other basis. In yet other embodiments, blood collection center personnel are provided with an inventory of available blood products and determine themselves which blood types are needed for which blood product based on blood collection center parameters.

Donors that enter a blood collection center are first screened for suitability by completing a health questionnaire and pre-screening interview with blood collection center personnel and then, if they are determined to be a candidate for blood donation, have a small quantity of blood drawn for blood type determination and for determination of hemoglobin/hematocrit. Hemoglobin/hematocrit is determined pre-donation to avoid drawing blood from anemic patients who may be harmed by removal of blood.

Blood type determination can be performed by any methods which rapidly (in less than 5 min or less than 1 min) and accurately types blood. In another embodiment, the blood type determined is ABO group and/or Rh(D) blood type. In another embodiment, the blood type determination is conducted with the blood type determination system described herein.

Once the blood of the candidate donor has been typed, the blood collection center personnel compares that blood type of the candidate donor with the inventory or analysis available at the blood collection center and determines if the candidate blood donor blood type is needed or desirable by the blood collection center. If the blood type is needed for whole blood or RBCs, the candidate blood donor enters the blood collection process and blood is collected according to standard procedures. If the candidate blood donor's blood type is not needed for blood (whole blood or RBCs), but is needed for platelets or plasma, then the donor is asked to donate platelets and/or plasma by apheresis. If the donor's blood type is not needed for any product, the donor is thanked and allowed to leave without donating blood or blood product(s). If desired, the candidate donor's contact information is retained by the blood collection center and if, or when, the candidate's blood type is needed for a particular product, the donor can be recalled to the blood collection center to collect that product.

Blood typing is used to determine the presence of specific clinically-important antigens on the RBC surface. These include, but are not limited to, A antigen and B antigen (collectively known as the ABO system), D antigen, and other RBC antigens including C, E, K and k (known as the Kell system), and Fya and Fyb (known as the Duffy system), etc. Usually, each of these antigens is tested for in an individual test/reaction.

Disclosed herein is a method for the rapid pre-donation screening of candidate blood donors and a blood type determination system based on the known observation that antibody-based typing reagents of the IgM class will cause agglutination (clumping) of red blood cells (RBCs) at room temperature if given enough time and coaptation (a decrease in the relative zeta-potential).

The antibodies of the blood type determination system disclosed herein are generally IgM. IgM antibodies have ten antigen binding sites per molecule and the IgM molecule is large enough to span the distance between RBCs, so that when the IgM antibody and reactive RBCs are mixed, the RBCs clump together (agglutinate). If the sample does not contain RBC antigens which are reactive with the particular IgM antibody, then the RBCs will not agglutinate.

In one embodiment disclosed herein, the blood type determination system comprises individual reaction vessels containing a filter matrix. Donor RBCs are added into the reaction vessels with an IgM antibody reactive with one of the A, B, or D antigens. Each reaction vessel provides a test environment for at least one blood group antigen.

Commercially available antibody blood typing reagents against A, B and D antigens may be used in small quantities at an appropriate dilution in saline. In one embodiment, a single drop of a suspension is suitable. The concentration of blood typing reagent is determined according to methods well known to persons of ordinary skill in the art according to established methods of determining antibody dilutions. With the application of a short centrifugation, agglutinated RBCs (a positive result) will not traverse the filter matrix, while non-agglutinated RBCs (a negative result) will traverse the filter matrix. The blood type of a particular donor sample can then be determined by visualization of whether the donor RBCs were retained above the filter matrix or pelleted in the bottom of the reaction vessel.

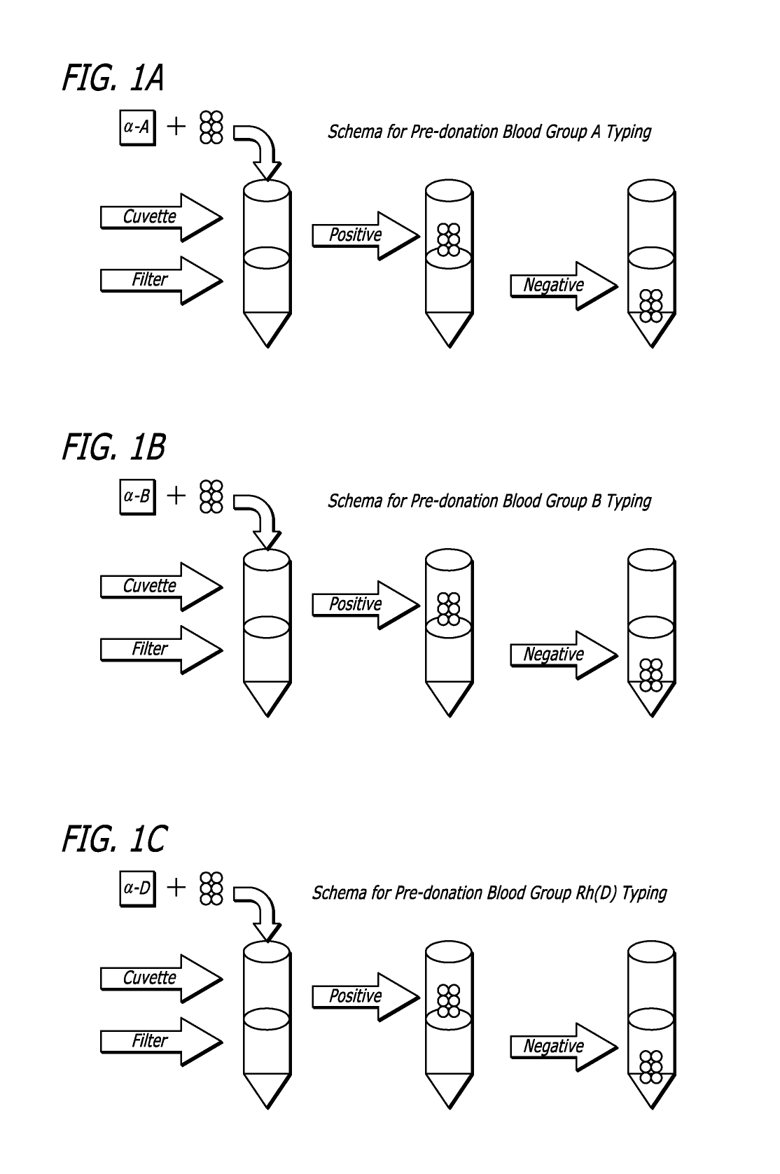

In one embodiment, depicted in FIG. 1A, an assay is performed wherein donor RBCs are mixed with an IgM antibody specific for blood group A antigen. If the donor RBCs express the A antigen, the RBCs will agglutinate and be prevented from entering the filter matrix during centrifugation and can be seen with the naked eye to be retained by the filter. Conversely, if the donor RBCs do not express the A antigen, the donor RBCs will not agglutinate and during centrifugation will pass through the filter material to the bottom of the reaction vessel.

In another embodiment, depicted in FIG. 1B, an assay is performed wherein donor RBCs are mixed with an IgM antibody specific for blood group B antigen. If the donor RBCs express the B antigen, the RBCs will agglutinate and be prevented from entering the filter matrix during centrifugation and can be seen with the naked eye to be retained by the filter. Conversely, if the donor RBCs do not express the B antigen, the donor RBCs will not agglutinate and during centrifugation will pass through the filter material to the bottom of the reaction vessel.

In another embodiment, depicted in FIG. 1C, an assay is performed wherein donor RBCs are mixed with an IgM antibody specific for blood group D antigen. If the donor RBCs express the D antigen, the RBCs will agglutinate and be prevented from entering the filter matrix during centrifugation and can be seen with the naked eye to be retained by the filter. Conversely, if the donor RBCs do not express the D antigen, the donor RBCs will not agglutinate and during centrifugation will pass through the filter material to the bottom of the reaction vessel.

In one embodiment, the reaction vessels comprise test tubes, cuvettes or wells. The tubes, cuvettes or wells can be any size appropriate to contain the filter material, antibodie(s) and donor RBCs. Furthermore, the tubes, cuvettes or wells can be configured as individual units or arranged together on a card or plate. One system will include one or more reaction vessels to test all or one of blood group antigens A, B and D as well as appropriate control vessels. There may be additional reaction vessels provided for preparing a test dilution or test suspension. The vessels may contain the filter matrix and/or industrially prepared reaction solutions.

The blood type determination system may be made in various ways. For example, small tubes may be glued to a card or may form an integral part of the card, in the manner of blister packaging. The filter matrix may be hermetically enclosed in these tubes in a predetermined quantity by the manufacturer, in which case the tubes may be sealed by means of a welded-on film. Alternatively, the blood type determination system can be manufactured with the filter matrix and appropriate antibodies pre-packaged in the reaction vessels.

In one embodiment of the disclosed blood type determination system, the filter matrix comprises an inert material and a plurality of pores. The pore size of the filter material may be varied, according to the various embodiments disclosed herein wherein the pores are sized to substantially retain the agglutinated RBCs such that the unreacted RBCs pass through the pores and accumulate in the bottom of the vessel. For example, pores of the filter material may be of a size ranging from approximately 5 microns to approximately 50 microns. The size of the pores of the filter material will depend on the application of the reaction vessel containing the filter matrix.

If it is desired that the filter matrix be used to retain, for example, agglutinated RBCs while allowing individual red blood cells (non-agglutinated) to pass through the pores of the filter, the range of pore sizes is between approximately 5 microns to approximately 40 microns. While other pore sizes may be used, in one embodiment, the pore size ranges from approximately 5 microns to approximately 8 microns.

The thickness of the filter matrix may also vary in different embodiments, depending upon the application of the filter. For example, the thickness of the filter matrix may range from approximately 3 microns to approximately 5 mm. In one embodiment the filter matrix is between approximately 3 microns to approximately 100 microns. Optimally, the thickness of the filter matrix is between approximately 10 microns and approximately 75 microns.

The filter material used for the filter matrix may be any material in the various embodiments of the reaction vessel, such as an inert material that includes a plurality of pores. The filter material may be varied, depending on the application of the filter matrix. If the function of the filter matrix is to retain agglutinated RBCs, while allowing individual RBCs to pass through, then the filter material may be, for example, but not limited to, a polyester mesh, a nylon mesh, or a polycarbonate track-etched membrane. A filter material of this type is manufactured by and commercially available from Sefar, Inc. in Kansas City, Mo.

In another embodiment, the filter matrix is comprised of a size exclusion material. The size exclusion material is typically based on cross-linked polymers such as agarose, polyacrylamide, polydextran or styrene-divinylbenzene polymers such as Sephadex®, Sepharose® or Sephacryl® (Pharmacia AB) or Biogel® (BioRad). Porous silica gel or glass can also be used as a size exclusion material. The size exclusion material is sized such that particles above a defined size do not enter the material while particles below the defined size pass through the material. For exclusion of agglutinated red blood cells, the size exclusion material should exclude any particles larger than 5-8 microns in size.

In another embodiment, the filter matrix is comprised of substantially noncompressable microparticles. By “substantially noncompressable” is meant resistant to change in shape or size that may be caused by the exertion of force to the matrix, such as centrifugal force, magnetic force, electrical force, hydrostatic pressure, force by negative or positive pressure, and the like, or storage for long periods of time with normal gravitational force. The particles may be of any shape as long as the movement of nonagglutinated RNCs is not impeded by irregularities, and so on. The size of the particles may vary considerably according to the particular binding ligands involved in the agglutination assay. One skilled in the art will understand that agglutinated RBCs should be retained on top of the matrix while non-agglutinated RBCs and reagents travel through the matrix to the bottom. However, in the case of agglutination of red blood cells, preferable matrix microparticle size ranges are generally from about 50 microns to about 300 microns, and more preferably about 50 microns to about 200 microns, and most preferably about 50 microns to about 150 microns. Certain size exclusion materials are comprised of substantially noncompressable microparticles.

Suitable noncompressable materials for use herein comprise various silicon dioxide compounds, including glass and sand, metal compounds as long as they are light enough in color to permit visual observation of agglutination if desired, various plastic compounds, and the like. Exemplary of the above materials are such commercially available polystyrene beads as those that may be obtained from Polysciences, Inc.; sea sand, available from Fisher or Mallinkrodt; cellulose particles, such as those available from Whatman; and glass beads such as those available from Jaygo Incorporated (from Dragonite) and Hüls Petrach Systems.

Although sedimentation can be brought about by allowing the vessels to stand and taking advantage of the force of gravity, procedures that facilitate the desired sedimentation after only a short time and under controlled conditions can also be used. Such procedures include without limitation centrifugation and vacuum-assisted filtration.

In one embodiment, the rapid blood type determination system further comprises a centrifuge. It should be understood that any type of centrifuge system known and used by those skilled in the art may be used as the centrifuge. For example, a typical centrifuge manufactured by and commercially available from Beckman Coulter, Inc. (Fullerton, Calif.) may be used in accordance with one embodiment disclosed herein so long as the centrifuge is modified to hold the reaction vessels.

In another embodiment, the rapid blood type determination system further comprises a vacuum apparatus. The reaction vessels are placed in the apparatus and a vacuum is applied to draw the unreacted RBCs through the filter.

In other embodiments disclosed herein, kits are provided for the pre-donation determination of blood type. The kits include antibodies and reaction vessels for determination of at least one blood group antigen along with appropriate control reaction vessels and antibodies. The kits may also include lancets, pipettes and other disposable supplies for performing the test along with written instructions. In one embodiment, the kits comprise reaction vessels and reagents for determining A, B and Rh(D) antigens.

### EXAMPLES

### Example 1

### Pre-Donation Determination of Blood Type to Select for Desired Blood Type Donors

A candidate donor desires to donate blood and visits a local blood collection center. After completion of a health screening questionnaire, it is determined by blood collection center personnel that the donor is an acceptable candidate. A small volume of blood is obtained from the candidate donor and the blood sample is processed to determine the blood type as well as the hemoglobin/hematocrit.

The blood collection center personnel then refers to a chart which determines which blood products should be collected from a candidate donor with the determined blood type. The blood type of the candidate donor is in short supply for red cells and/or is in high demand and the candidate donor is then passed through to the blood donation process.

### Example 2

### Pre-Donation Determination of Blood Type to Eliminate Collection of Unneeded Blood Types

A candidate donor desires to donate blood and visits a local blood collection center. After completion of a health screening questionnaire, it is determined by blood collection center personnel that the donor is an acceptable candidate. A small volume of blood is obtained from the candidate donor and the blood sample is processed to determine the blood type as well as the hemoglobin/hematocrit.

The blood collection center personnel then refers to a chart which determines what blood products should be collected from a potential donor with the determined blood type. If no blood is needed of the candidate donor type, the candidate donor is thanked for their time and released from the blood donation process. The candidate donor's contact information is retained in the blood collection center database in case it is necessary to recall the donor when the blood type is needed in the future

### Example 3

### Pre-Donation Determination of Blood Type to Direct Candidate Donors to Apheresis

A candidate donor desires to donate blood and visits a local blood collection center. After completion of a health screening questionnaire, it is determined by blood collection center personnel that the donor is an acceptable candidate. A small volume of blood is obtained from the candidate donor and the blood sample is processed to determine the blood type as well as the hemoglobin/hematocrit.

The blood collection center personnel then refers to a chart which determines what blood products should be collected from a potential donor with the determined blood type. The blood type of the candidate donor is not needed for red cells or whole blood. The candidate's blood type is, however, needed for platelets or plasma donation. The candidate donor is asked to make an apheresis donation of the needed type.

### Example 4

### Pre-Donation Determination of Blood Type

With regard to Examples 1-3, the blood type of the candidate donor is determined as follows: A donor blood sample is obtained and a volume of whole blood is applied to a plurality of reaction vessels or wells and to each reaction vessel or well is also added a volume of a reagent to determine a single blood group antigen, such as an IgM antibody specific for the blood group antigen. Optionally, in additional reaction vessels or wells controls reagents can be added. At least two control reagents can be used. The first control comprises antigen-positive RBCs that will also agglutinate with the particular antibody in the vessel or well when the donor RBCs did not result in a reaction. The second control reagent is specific for an antigen that is not present on RBCs which will not agglutinate any type blood and serves as a negative control. After an incubation time of 0.1-10 minutes, such as less than 1 minute, the reaction vessels or wells are centrifuged. Alternatively, the reaction vessels are not centrifuged and the agglutinated RBCs pass through, or are retained by, the filter due to the force of gravity. If the donor blood RBCs express an antigen, the RBCs will agglutinate and be prevented from entering the filter matrix and can be seen with the naked eye to be retained by the filter and have the same result as the positive control. Conversely, if the donor RBCs do not express the antigen, the donor RBCs will not agglutinate and will pass through the filter material to the bottom of the reaction vessel and have the same result as the negative control.

The blood type of the candidate donor blood sample is determined by determining whether the RBCs agglutinate in the presence of each antibody to antigen tested to generate a blood type phenotype.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method for increasing the efficiency and reducing cost of blood donation, processing and storage comprising:
a) pre-determining at least one blood type needed by a blood collection center;
b) determining a blood type of a blood sample from at least one candidate blood donor; and
c) acting upon the results of the determining step by an action selected from the group consisting of (i) accepting the candidate blood donor for blood donation if the blood type of the candidate donor corresponds to the blood type needed in step (a), (ii) rejecting the candidate blood donor if the candidate donor's blood type is not needed in step (a), or (iii) referring the candidate donor to donation of an alternate blood product independent of the blood type of the candidate donor.

2. The method according to claim 1, further comprising the step of pre-determining a blood product needed by a blood collection center.

3. The method according to claim 1, wherein the blood type is an ABO blood group type.

4. The method according to claim 1, wherein the blood type is an Rh(D) blood group type.

5. The method according to claim 1, wherein the blood product is whole blood or red blood cells.

6. The method according to claim 1, wherein the alternate blood product is platelets or plasma.

7. The method according to claim 1, wherein said determining step comprises an antibody-based assay.

8. The method according to claim 7, wherein said antibody is an immunoglobulin M (IgM) antibody.

9. A method for increasing the efficiency and reducing cost of blood donation, processing and storage comprising:
a) pre-determining blood types needed by a blood collection center;
b) obtaining a blood sample from at least one candidate blood donor;
c) mixing at least a portion of said blood sample with reagents for determining blood types to form a reaction mixture;
d) processing said reaction mixture for a time and at a temperature sufficient for a reaction to occur;
e) detecting said reaction or absence thereof;
f) correlating said reaction or absence thereof with a blood type; and
g) acting upon the results of the correlating step by an action selected from the group consisting of (i) accepting the candidate blood donor for blood donation if the blood type of the candidate donor corresponds to the blood type needed in step (a), (ii) rejecting the candidate blood donor if the candidate donor's blood type is not needed in step (a), or (iii) referring the candidate donor to donation of an alternate blood product independent of the blood type of the candidate donor, such that the efficiency of the blood donation process is increased and the costs of blood donation, processing and storage are reduced.

10. The method according to claim 9, further comprising the step of pre-determining a blood product needed by a blood collection center.

11. The method according to claim 9, wherein the blood product is whole blood or red blood cells.

12. The method according to claim 9, wherein the alternate blood product is platelets or plasma.

13. A method of determining blood type prior to blood donation using a blood type determination system, said method comprising the steps of:
(a) obtaining a sample of blood from a candidate blood donor;
(b) mixing at least a portion of said blood sample with an antibody specific for a blood group antigen;
(c) determining the blood type of said blood based on the agglutination of said red blood cells by said antibody;
(d) repeating steps (b) and (c) for each blood group antigen to be tested; and
(e) developing a blood type phenotype for said candidate donor based upon the blood type determined in step (d).

14. The method according to claim 13 wherein said blood type is an ABO blood group type.

15. The method according to claim 13 wherein said blood type is an Rh(D) blood group type.

16. The method according to claim 13 wherein said antibody is an immunoglobulin M (IgM) antibody.

17. The method according to claim 13 wherein said antibody is a control antibody.

18. The method according to claim 13 wherein said blood determination system comprises:
a plurality of reaction vessels, wherein said reaction vessels further comprise a filter matrix;
at least antibody specific for a blood group antigen; and
optionally, a centrifuge.

19. The method according to claim 18 wherein said plurality of reaction vessels are grouped together on a card or plate.

20. The method according to claim 18 wherein said filter matrix is an inert material with pores or a size exclusion material.

21. The method according to claim 18 wherein said blood type determination system optionally further comprises negative and/or positive control reaction vessels.

22. A method of determining blood type prior to blood donation using a blood type determination system, said method comprising the steps of:
(a) obtaining a sample of blood from a candidate blood donor;
(b) mixing at least a portion of said blood sample with an antibody specific for a blood group antigen;
(c) applying said mixture to a reaction vessel, wherein said reaction vessel comprises a filter matrix;
(d) centrifuging said reaction vessel to facilitate the passage of non-agglutinated red blood cells through said filter matrix to the bottom of said reaction vessel; and
(e) determining the blood type of said blood based on the agglutination of said red blood cells by said antibody;
(f) repeating steps (b)-(e) for each blood group antigen to be tested; and
(g) developing a blood type phenotype for said candidate donor based upon the blood type determined in step (e).

23. A system for increasing the efficiency and reducing cost of blood donation, processing and storage in a blood bank or blood collection center comprising:
an inventory of blood and/or blood component units available in said blood bank or blood collection center;
a kit comprising a blood type determination system for determining the blood type of a candidate donor prior to donation; and
means for determining if the blood type of the candidate donor is desirable in said inventory.
